(19) United States
(12) Patent Application Publication
(10) Pub. No.: US 2015/0119607 A1
(43) Pub. Date: Apr. 30, 2015

(54) LIGNOCELLULOSIC CONVERSION PROCESS WITH TISSUE SEPARATION

(71) Applicant: BP Corporation North America Inc., Naperville, IL (US)

(72) Inventors: Joseph B. Binder, Berkeley, CA (US); Jacob Borden, Lake Charles, LA (US); Micheal L. Chappell, Encinitas, CA (US)

(73) Assignee: BP Corporation North America Inc., Naperville, IL (US)

(21) Appl. No.: 14/390,673

(22) PCT Filed: Apr. 30, 2013
(86) PCT No.: PCT/US13/38783

§ 371 Date: Oct. 3, 2014

Related U.S. Application Data
(60) Provisional application No. 61/640,503, filed on Apr. 30, 2012.

(51) Int. Cl. C12P 7/10; C12P 7/14; C12M 1/00; C07C 31/08

(52) U.S. Cl. CPC C12P 7/10; C07C 31/08; C12P 7/14; C12M 43/02; C12M 21/12; C12M 45/04; C12P 2201/00
USPC 568/840; 435/165; 435/162; 435/289.1

(57) ABSTRACT

Methods of producing renewable materials, such as biofuels, may include separating lignocellulosic feedstock into various fractions, pretreating at least one of the fractions, and further treating the pretreated fraction(s) to produce a renewable material. More particularly, an outer-most stalk tissue, or rind, of the lignocellulosic feedstock having the least-accessible carbohydrates can be separated from the leaves and pith of the feedstock. Then the easily-accessible leaves, pith, and sugars can be processed together, while the rind can either be processed separately to produce a renewable material, or turned into other products. In certain embodiments, a cane tissue fractionation system is included at a front end of a sugar mill.

### BACKGROUND

1. Technical Field

The invention relates to methods and systems directed to renewable materials and biofuels production. Aspects of the invention relate to separating lignocellulosic feedstock into various fractions for improved processing.

2. Discussion of Related Art

Lignocellulosic biomass contains sugars in multiple forms, such as soluble sugars, starches, hemicelluloses, and cellulose. Some sugars, such as soluble sugars, are relatively accessible for utilization. Other sugars, such as cellulose and hemicellulose, require multiple processing steps to prepare them for fermentation. Moreover, the ease of access depends on the plant tissue in which the sugars are found, with the soft inner pith easier to digest and the hard outer rind requiring harsher conditions to break down.

Processing of lignocellulosic biomass is challenging because the accessibility and processing conditions vary among the different forms of sugars. In some lignocellulosic processing schemes, harsh conditions are used to break down even the least accessible biomass sugars, and providing these harsh conditions throughout the process for all of the sugars requires increased capital and operating expenses. Also, processing carried out under harsh conditions requires that the soluble sugars present in the biomass be removed before further processing in order to avoid their degradation. The need to remove soluble sugars during the process further increases capital expenses.

Additionally, lignocellulosic feedstocks contain substantial quantities of water. The cost, power demand, and performance efficiency of certain equipment is variably impacted by hydraulic load. For instance, industrial boilers are sensitive to the moisture content of the combusted material. Altering the moisture content of the lignocellulosic feedstock, such as by preferentially converting fractions containing lower moisture content, could allow for preferential disposition of feedstock fractions with varying moisture content.

There is a need and desire for more economical processes and systems for processing lignocellulosic biomass, particularly in the production of renewable materials such as biofuels.

### SUMMARY

The invention is directed to methods and systems for producing biofuels and other renewable materials, as well as biofuel component compositions made according to such methods. Compared to other schemes for processing of lignocellulosic biomass, the methods and systems described below may result in minimized capital, reduced operating expenses, and increased yield.

According to some embodiments, a method of producing a renewable material may be achieved by separating lignocellulosic feedstock into two or more fractions, such as three, four, five, or even more fractions. For example, the various fractions may include rind, pith, leaves, juice, wax cut, and/or trash. As another example, one or more of the various fractions may be enriched in cellulose, hemicellulose, and/or lignin.

The lignocellulosic feedstock used herein may include cellulose, hemicellulose, and/or lignin. For example, the lignocellulosic feedstock may include energycane, sugarcane, miscanthus, napier grass, elephant grass, sweet sorghum, arundo, switchgrass, hybrids thereof and/or combinations thereof. As another example, the lignocellulosic feedstock may include grasses, legumes, forbs, hardwoods, softwoods, municipal solid waste, paper mill residue, forest litter, fruit waste, citrus waste, and/or agricultural residues.

In certain embodiments, an automated separation device may be used to separate the lignocellulosic feedstock.

In certain embodiments, the method also includes pretreating at least one of the fractions, and further treating the pretreated fraction or fractions to produce a renewable material. According to some embodiments, each of the fractions may be pretreated. Each of the fractions may be pretreated separately, or one or more of the fractions may be pretreated simultaneously with at least one other fraction.

In certain embodiments, juice may be extracted from the lignocellulosic feedstock prior to separating the lignocellulosic feedstock. Alternatively, juice may be extracted from the lignocellulosic feedstock subsequent to separating the lignocellulosic feedstock. In other embodiments, juice may not be substantially extracted from the separated lignocellulosic feedstock fraction prior to pretreatment. A juice fraction that is extracted may be sent to a sucrose production train within a biorefinery.

Since the fractions may vary in terms of ease with which they may be broken down into sugars, various levels of harshness may be used in the pretreatment process. For example, a fraction comprising rind may be pretreated using a harsh or relatively more harsh pretreatment process. Conversely, a fraction comprising pith and/or leaves may be pretreated using a mild or relatively more mild pretreatment process. More particularly, a pith fraction may be pretreated by hydrolyzing the pith fraction with low or no acid at a temperature of at least 110° C., then the pith fraction may be hydrolyzed with an enzyme cocktail to produce sugars for fermentation. Similarly, a trash fraction may be pretreated by hydrolyzing the trash fraction with low or no acid at a temperature of at least 110° C., then the trash fraction may be hydrolyzed with an enzyme cocktail to produce sugars for fermentation.

According to some embodiments, the pretreatment may use an ionic liquid, an acid, a base, an enzyme, and/or water. For example, an acid used in the pretreatment may include an inorganic acid, an organic acid, a mineral acid, a Bronsted acid, and/or a Lewis acid. More specifically, an acid used in the pretreatment may include sulfuric acid, hydrochloric acid, sulfonic acid, phosphoric acid, nitric acid, acetic acid, lactic acid, formic acid, and/or oxalic acid. As another example, a base used in the pretreatment may include an inorganic base, an organic base, a mineral base, a Bronsted base, and/or a Lewis base. More specifically, a base used in the pretreatment may include ammonia, ammonium hydroxide, sodium hydroxide, potassium hydroxide, magnesium hydroxide, lime, calcium hydroxide, and/or calcium oxide. As yet another example, enzymes used in the pretreatment may include cellulases, glucanases, endoglucanases, exoglucanases, cellobiohydrolases, glucosidases, hemicellulases, esterases, acetylxylanesterases, pectinases, and/or the like. As a further example, facilitating proteins, such as expansins and/or swolleins, may be used in the pretreatment. Additionally, at least in some embodiments, pretreatment may be carried out on one or more of the fractions without heating. Additionally, at least one of the fractions may be burned to generate steam and/or electricity.

After separating the lignocellulosic feedstock into fractions, the fractions may be directed to various areas within a biorefinery. For example, a rind fraction may be sent to cane handling equipment for sugar recovery. A bagasse remainder may be sent to a boiler for power production, while sugar from the rind may either be sent to a sucrose recovery unit or to a fermentation unit. As another example, wax from a wax cut fraction may be sent to a wax recovery unit. Any fiber remainder may be burned.

According to some embodiments, at least one of the fractions may be converted to one or more sugars, such as sucrose, glucose, fructose, mannose, galactose, xylose, arabinose, various hexoses, various pentoses, cellobiose, and/or oligosaccharides. The sugar(s) may be converted into a renewable material. Examples of such renewable materials include ethanol, n-butanol, isobutanol, 2-butanol, fatty alcohols, isobutene, isoprenoids, triglycerides, lipids, fatty acids, lactic acid, acetic acid, propanediol, and/or butanediol. The renewable material may include material suitable for use as biofuels, blendstocks, chemicals, intermediates, solvents, adhesives, polymers, and/or lubricants. The renewable material may include one or more biofuel components, such as lipids, or an alcohol, namely ethanol, butanol, and/or isobutanol.

In certain embodiments, at least one of the fractions is converted to one or more sugars, and the sugar or sugars are converted into a renewable material suitable for use as a biofuel. The biofuel may include biofuel intermediates, gasoline, biogasoline, biogasoline blendstocks, diesel, biodiesel, green diesel, renewable diesel, biodiesel blend stocks, jet fuel, and/or kerosene. In some embodiments, at least one of the fractions is combusted.

According to some embodiments, a method of producing a biofuel component may be achieved by separating lignocellulosic feedstock into two or more fractions, pretreating at least one of the fractions, and further treating the at least one pretreated fraction to produce a biofuel component.

According to some embodiments, a lignocellulosic feedstock separated into a plurality of fractions may include between about 30% and about 85% of a first fraction that includes primarily rind by dry weight, and between about 15% and about 70% of a second fraction that includes primarily pith by dry weight. In certain embodiments, the first fraction may include between about 30% and about 50% cellulose by dry weight, and/or between about 15% and about 30% hemicellulose by dry weight, and/or between about 15% and about 25% lignin by dry weight. In certain embodiments, the second fraction may include between about 15% and about 40% cellulose by dry weight, and/or between about 9% and about 25% hemicellulose by dry weight, and/or between about 5% and about 19% lignin by dry weight.

According to some embodiments, a lignocellulosic feedstock separated into a plurality of fractions may include a first fraction enriched in polysaccharides, and a second fraction enriched in lignin.

According to some embodiments, a biofuel component composition may be derived from a lignocellulosic feedstock that was separated into a plurality of fractions, with between about 30% and about 85% of a first fraction that includes primarily rind by dry weight, and between about 15% and about 70% of a second fraction that includes primarily pith by dry weight, prior to converting one or more of the fractions into a biofuel component. The biofuel component may include an alcohol, such as ethanol and/or butanol.

According to some embodiments, a biorefinery for producing biofuels may include a tissue separation unit for separating tissues of a lignocellulosic feedstock, a pretreatment unit for treating at least some of the separated tissues of the lignocellulosic feedstock, and a conversion unit for producing a renewable material from the separated tissues. In certain embodiments, a biorefinery for producing biofuels may include a cane tissue fractionation system at a front end of the biorefinery, a sugar mill behind the cane tissue fractionation system, and a conversion unit for producing a renewable material.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 is a process flow diagram illustrating a conventional method for producing a renewable material.

FIG. 2 is a process flow diagram illustrating one embodiment of a method for producing a renewable material using tissue separation.

FIG. 3 is a process flow diagram illustrating another embodiment of a method for producing a renewable material using tissue separation.

FIG. 4 is a process flow diagram illustrating yet another embodiment of a method for producing a renewable material using tissue separation.

FIG. 5 is a diagram illustrating one embodiment of a tissue fractionation system installed in an existing sugar mill.

FIGS. 6-8 are graphical representations of data derived from Example 4.

FIG. 9 is a graphical representation of data derived from Example 6.

FIGS. 10 and 11 are graphical representations of data derived from Example 7.

### DETAILED DESCRIPTION

The invention is directed to methods and systems for producing biofuels and other renewable materials using a lignocellulosic conversion process that involves tissue separation, as well as biofuel component compositions made according to such methods.

As used herein, the term “renewable material” preferably refers to a substance and/or an item that has been at least partially derived from a source and/or a process capable of being replaced at least in part by natural ecological cycles and/or resources. Renewable materials may broadly include, for example, chemicals, chemical intermediates, solvents, adhesives, lubricants, monomers, oligomers, polymers, biofuels, biofuel intermediates, biogasoline, biogasoline blendstocks, biodiesel, green diesel, renewable diesel, biodiesel blend stocks, biodistillates, biochar, biocoke, renewable building materials, and/or the like. As a more specific example, the renewable material may include, without being limited to, any one or more of the following: methane, ethanol, n-butanol, isobutanol, 2-butanol, fatty alcohols, isobutene, isoprenoids, triglycerides, lipids, fatty acids, lactic acid, acetic acid, propanediol, butanediol. In certain embodiments, the renewable material may include one or more biofuel components. For example, the renewable material may include an alcohol, such as ethanol, butanol, or isobutanol, or lipids.

The term “biofuel” preferably refers to components and/or streams suitable for use as a fuel and/or a combustion source derived at least in part from renewable sources. The biofuel can be sustainably produced and/or have reduced and/or no net carbon emissions to the atmosphere, such as when compared to fossil fuels. According to some embodiments, renewable sources can exclude materials mined or drilled. In some embodiments, renewable resources can include single cell organisms, multi-cell organisms, plants, fungi, bacteria, algae, cultivated crops, non-cultivated crops, timber, and/or the like. Biofuels can be suitable for use as transportation fuels, such as for use in land vehicles, marine vehicles, aviation vehicles, and/or the like. More particularly, the biofuels may include gasoline, diesel, jet fuel, kerosene, and/or the like. Biofuels can be suitable for use in power generation, such as raising steam, exchanging energy with a suitable heat transfer media, generating syngas, generating hydrogen, making electricity, and/or the like.

The term “fraction,” as used herein, preferably refers to one of the separable or separated constituents of a substance, such that all of the fractions together render the substance whole.

Lignocellulosic preferably broadly refers to materials containing cellulose, hemicellulose, lignin, juice, and/or the like, such as may be derived from plant material and/or the like. Lignocellulosic material may include any suitable material, such as sugarcane, sugarcane bagasse, energycane, energycane bagasse, rice, rice straw, corn, corn stover, maize, maize stover, wheat, wheat straw, sorghum, sorghum stover, sweet sorghum, sweet sorghum stover, arundo, cotton remnant, sugar beet, sugar beet pulp, soybean, rapeseed, jatropha, switchgrass, miscanthus, napier grass, other grasses, and hybrids of any of these materials. Lignocellulosic material may also include, in general, grasses legumes, forbs, cacti, timber, wood chips, softwoods such as pine and poplar, hardwoods such as eucalyptus, oak, and hickory, forest litter, wood waste, sawdust, paper, paper mill residue, paper waste, fruit waste, citrus waste, agricultural waste, municipal solid waste, any other suitable biomass material, and/or the like. According to some embodiments, the methods and systems described herein may apply to lignocellulosic materials other than or excluding corn, corn stover, maize, and maize stover.

Lignin preferably broadly refers to a biopolymer that may be part of secondary cell walls in plants, such as a complex highly cross-linked aromatic polymer that may covalently link to hemicellulose.

Hemicellulose preferably broadly refers to a branched sugar polymer composed mostly of pentoses, such as with a generally random amorphous structure and typically may include up to hundreds of thousands of pentose monomers.

Cellulose preferably broadly refers to an organic compound with the formula (C₆H₁₀O₅)_{z} where z includes any suitable integer. Cellulose may include a polysaccharide with a linear chain of several hundred to over ten thousand hexose monomers and may have a high degree of crystalline structure or may be amorphous, for example.

Juice preferably refers broadly to water and water-soluble molecules. Water may include bound and free water. Water-soluble molecules may include soluble sugars, sucrose, fructose, glucose, proteins, organic acids, minerals, salts, and the like.

As explained above, lignocellulosic biomass contains sugars in multiple forms, such as soluble sugars, starches, hemicellulose, and cellulose. Some of these sugars are relatively accessible, while others require multiple processing steps to prepare them for fermentation. By separating the lignocellulosic feedstock into multiple fractions based on the processing conditions required for each fraction, processes for producing renewable materials, such as biofuels, can be streamlined, resulting in capital minimization, reduced operating expenses, and increased yield.

In conventional processing of lignocellulosic biomass, the feedstock is typically pretreated under relatively harsh conditions in order to break down even the toughest of the lignocellulosic materials, such as the rind.

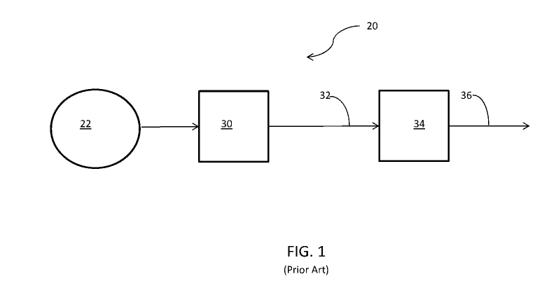

FIG. 1 illustrates an example of conventional lignocellulosic biomass processing (Prior Art). More particularly, in the system 20 illustrated in FIG. 1, the lignocellulosic feedstock 22 is fed to a pretreatment unit 30, where the feedstock 22 undergoes a relatively harsh pretreatment in order to break down all of the materials included in the feedstock 22. The pretreated feedstock 32 is then fed to a conversion unit 34, such as a saccharification unit and/or a fermentation unit to produce a renewable material 36.

According to some embodiments herein, a more economical method (reduced capital or operating costs) for producing a renewable material may be achieved by separating lignocellulosic feedstock into two or more fractions, pretreating at least one of the fractions, and further treating the pretreated fraction or fractions to produce a renewable material. For instance, the juice may be processed along with the lignocellulosic leaves and pith and, because these components are easier to break down into sugars than the rind, they can be pretreated mildly and saccharified and fermented in high yield together. Mild pretreatment conditions may also result in reduced capital costs and/or reduced operating expenses compared to equipment required to carry out harsh pretreatment.

The cellular difference between pith and rind is that pith contains a majority of parenchyma cells with a small percentage of vascular bundles, namely pith contains more than 50% parenchyma cells on a dry weight basis and less than 50% vascular bundles on a dry weight basis. Rind, on the other hand, contains a mixture of different cell types, including epidermis, sclerenchyma, parenchyma, and vascular bundles, namely rind contains less than 50% parenchyma cells on a dry weight basis.

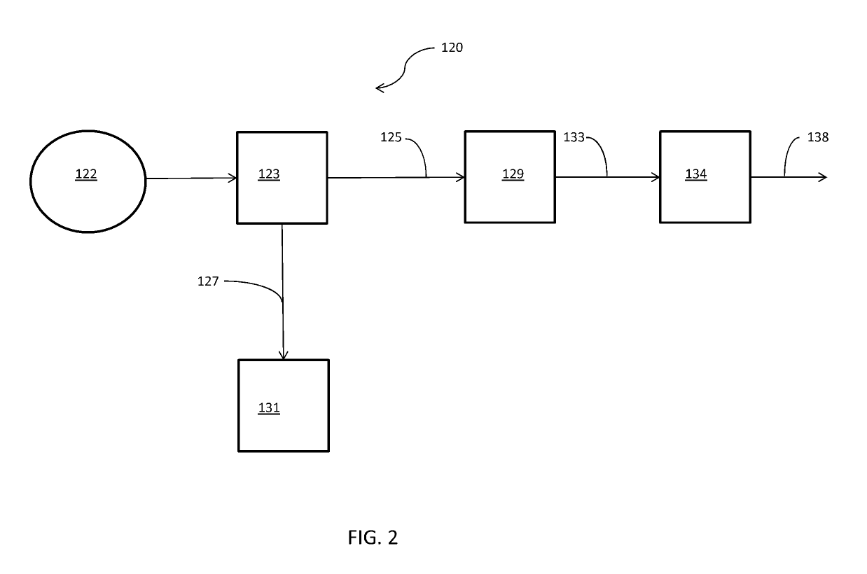

FIG. 2 illustrates a method for producing a renewable material, such as a biofuel, according to one embodiment, using a lignocellulosic conversion process involving tissue separation. In the system 120 illustrated in FIG. 2, the lignocellulosic feedstock 122 is fed to a tissue separation unit 123, which separates tissues of the lignocellulosic feedstock 122 into two or more fractions. As illustrated in the system of FIG. 2, a first fraction 125 including materials that are relatively easy to process, such as pith, leaves, and/or sugar, is fed to a pretreatment unit 129 while a second fraction 127 including materials that are more difficult to process, such as rind, is fed to an alternative-use unit 131. The pretreated first fraction 133 is then fed to a conversion unit 134, such as a saccharification unit and/or a fermentation unit, to produce the renewable material 138. Meanwhile, because the rind is the highest in lignin and most difficult to break down, the second fraction 127 in the alternative-use unit 131 may either be burned to generate steam and electricity, which may be sold to the grid to improve greenhouse gas balance, or used for quality particle board, fiber products, or waxes, for example. Prior to burning the second fraction 127 or applying the second fraction 127 to other uses, such as producing particle board, fiber products, or waxes, any remaining sugars may first be extracted from the rind.

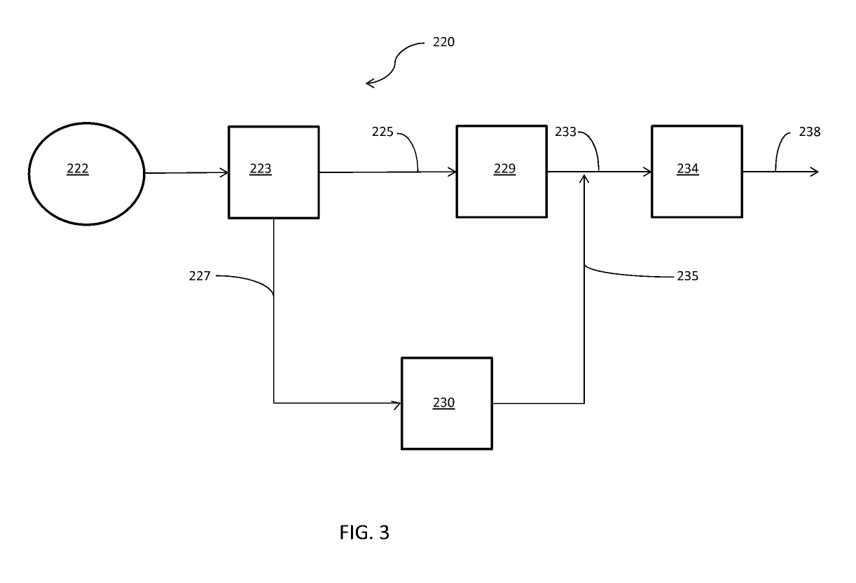

FIG. 3 illustrates another embodiment of a method for producing a renewable material, such as a biofuel, using a lignocellulosic conversion process involving tissue separation. The system 220 illustrated in FIG. 3 is very similar to the system 120 illustrated in FIG. 2, but instead of feeding the second fraction 127 to an alternative-use unit 131, the second fraction is pretreated and subsequently rejoined with the pretreated first fraction for conversion to the renewable material. In particular, in FIG. 3, the lignocellulosic feedstock 222 is fed to a tissue separation unit 223, which separates tissues of the lignocellulosic feedstock 222 into two or more fractions. As illustrated in the system of FIG. 3, a first fraction 225 including materials that are relatively easy to process, such as pith, leaves, and/or sugar, is fed to a mild pretreatment unit 229 while a second fraction 227 including materials that are more difficult to process, such as rind, is fed to a harsh pretreatment unit 230. The pretreated second fraction 235 is then combined with the pretreated first fraction 233, and both the pretreated first fraction 233 and the pretreated second fraction 235 are then fed to a conversion unit 234, such as a saccharification unit and/or a fermentation unit, to produce the renewable material 238. While the method illustrated in FIG. 3 requires more capital in terms of equipment used for pretreating the second fraction 227, and greater operating expenses due to operation of the harsh pretreatment unit 230, compared to the method illustrated in FIG. 2, the method in FIG. 3 generates a greater yield of the renewable material 238.

Example 1 below compares ethanol production of three different methods based on the methods illustrated in FIGS. 1-3.

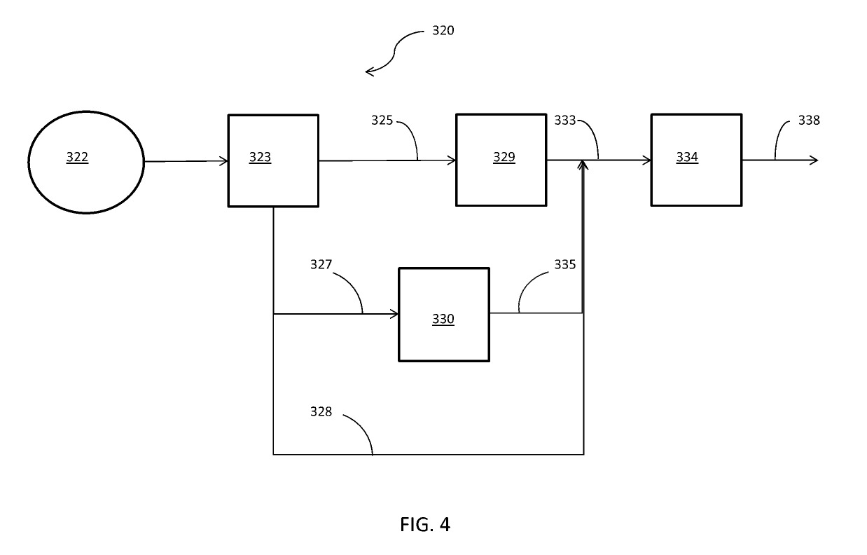

FIG. 4 illustrates yet another embodiment of a method for producing a renewable material, such as a biofuel, using a lignocellulosic conversion process involving tissue separation. The system 320 illustrated in FIG. 4 is very similar to the system 220 illustrated in FIG. 3, but instead of separating the lignocellulosic feedstock 222 into two fractions, the lignocellulosic feedstock 322 is fed to a tissue separation unit 323, which separates tissues of the lignocellulosic feedstock 322 into three fractions. As illustrated in the system of FIG. 4, a first fraction 325 including materials that are relatively easy to process, such as pith and leaves, is fed to a mild pretreatment unit 329, a second fraction 327 including materials that are more difficult to process, such as rind, is fed to a harsh pretreatment unit 330, and a third fraction 328 including soluble sugars, for example, is separated from the first and second fractions 325, 327. In certain embodiments, the juice may be removed from the lignocellulosic feedstock 322 at the outset to avoid degradation of the soluble sugars therein, as may occur in certain pretreatment processes. However, as exemplified in FIGS. 2 and 3, the mild conditions employed to pretreat the first separated fraction may preclude the need for juice separation to prevent soluble sugar degradation. Thus, in some embodiments, juice may not be substantially extracted from the separated lignocellulosic feedstock fraction prior to pretreatment.

Referring again to FIG. 4, the pretreated second fraction 335 and the third fraction 328 are combined with the pretreated first fraction 333, and all three fractions 325, 327, 328 are fed to a conversion unit 334, such as a saccharification unit and/or a fermentation unit, to produce the renewable material 338. Alternatively, as described with respect to FIG. 2, any one or more of the fractions may be separated from the feedstock and subsequently used for purposes other than being rejoined with the other fraction or fractions and converted into a renewable material.

An automated separation device, such as the KTC Tilby Cane Separation System, available from KTC Tilby Ltd. of Saanichton, British Columbia, may be used in conjunction with a trash separation/cane cleaning system to separate the lignocellulosic feedstock into any suitable number of fractions. Other systems for separating rind may be used as well, such as systems used for depithing in the production of quality fiber from bagasse.

For a grass crop such as sugarcane, maize, napiergrass, or sorghum, trash includes brown, senesced leaves; green, living leaves; and the apical internodes or growing point region at the top of the stalk.

In certain embodiments, the method may include both tissue separation and juice separation operations. In these embodiments, juice may be extracted from the lignocellulosic feedstock either before or after separating the lignocellulosic feedstock into two or more fractions.

The lignocellulosic feedstock may be separated into any suitable number of fractions, such as two, three, four, or five fractions. The lignocellulosic feedstock may be separated into even more fractions, in certain embodiments. According to some embodiments, one or more fractions may be enriched in water while one or more fractions may be depleted in water. The fraction or fractions depleted in water may be combusted.

For example, the lignocellulosic feedstock may be separated to generate a fraction that is enriched in cellulose, and/or a fraction that is enriched in hemicellulose, and/or a fraction that is enriched in lignin, and/or a fraction that is enriched in polysaccharides. In some embodiments, the lignocellulosic feedstock may be separated to generate a first fraction that is enriched in cellulose, and/or a first fraction that is enriched in hemicellulose, and/or a first fraction that is enriched in lignin, and/or a first fraction that is enriched in polysaccharides.

According to some embodiments, the lignocellulosic feedstock may be separated into at least two fractions, with the first fraction including between about 30% and about 85% primarily rind by dry weight, and the second fraction including between about 15% and about 70% primarily pith by dry weight. More particularly, the first fraction may include between about 30% and about 50% cellulose by dry weight, between about 15% and about 30% hemicellulose by dry weight, and between about 15% and about 25% lignin by dry weight, and the second fraction may include between about 15% and about 40% cellulose by dry weight, between about 9% and about 25% hemicellulose by dry weight, and between about 5% and about 19% lignin by dry weight.

According to some embodiments, each of the fractions may be pretreated. One or more, or even all of the fractions may be pretreated. The fractions may each be pretreated separately. Alternatively, two or more of the fractions may be pretreated together, either before or after separation.

As explained above, a harsh pretreatment process can be used to break down hard-to-digest components, such as fractions that include rind, while a mild pretreatment process can be used to break down components that are more easily digested, such as fractions that include pith and/or leaves. An empirical measure of pretreatment “harshness” is the “Combined Severity Factor,” which is calculated according to the following equation, wherein time is expressed in minutes and temperature is expressed in degrees Celsius: Log₁₀(t×exp((T-100)/14.75))−pH (Schell, D. F.; Farmer, J.; Newman, M.; McMillan, J. D. Applied Biochemistry and Biotechnology, 2003, 105-108, pp 69-85). Similarly, the “Log Severity Factor” is calculated according to the following equation, wherein time is expressed in minutes and temperature is expressed in degrees Celsius: Log₁₀(t×exp((T-100)/14.75)) (Kazi, K. M. F.; Jollez, P.; Chornet, E. Biomass and Bioenergy, 1998, 15, pp 125-141). However, these measurements are useful only for water-only and/or acid-catalyzed pretreatments.

The arbitrary terms “harsh” and “mild” are relative terms that depend greatly on the nature of the pretreatment (acid, base, solvent, etc.) and on the context of the process (enzymes, organisms, etc.). In embodiments involving two or more fractions that are pretreated separately, a “harsh” pretreatment process would have a higher log severity factor than a “mild” pretreatment process.

For dilute acid pretreatment, harsh pretreatment may include pretreatment with a combined severity factory of greater than 1.35 and mild pretreatment may include pretreatment with a combined severity factor of less than 1.35, as measured at 150° C., for 30 minutes, in 0.25% H₂SO₄. For hot water pretreatment and other pretreatments besides those employing dilute acid, harsh pretreatment may include pretreatment with a log severity factor of greater than 3 and mild pretreatment may include pretreatment with a log severity factor less than 3. For some feedstocks, harsh pretreatment may include pretreatment with a log severity factor of greater than 4 and mild pretreatment may include pretreatment with a log severity factor less than 4.

The pretreatment processes may include use of an ionic liquid, an acid, a base, an enzyme, and/or water. For example, the acid may include an inorganic acid, an organic acid, a mineral acid, a Bronsted acid, and/or a Lewis acid. More particularly, the acid may include sulfuric acid, hydrochloric acid, sulfonic acid, phosphoric acid, nitric acid, acetic acid, lactic acid, formic acid, and/or oxalic acid. When the pretreatment process involves using a base, the base may include an inorganic base, an organic base, a mineral base, a Bronsted base, and/or a Lewis base. More particularly, the base may include ammonia, ammonium hydroxide, sodium hydroxide, potassium hydroxide, magnesium hydroxide, lime, calcium hydroxide, and/or calcium oxide. In certain embodiments, the pretreatment may be carried out without heating the fraction or fractions. Conversely, in certain embodiments, the application of heat, such as in the form of steam, may be used in the pretreatment process.

During the processes described herein, one or more of the fractions may be converted to a sugar or sugars. The sugars may include, but are not limited to, sucrose, glucose, fructose, mannose, galactose, xylose, arabinose, hexose, pentose, cellobiose, and/or oligosaccharides. Any one or more of these sugars may be converted into a renewable material.

According to some embodiments, a cane fractionation system, such as the KTC Tilby Cane Separation System mentioned above, may be added at a front end of an existing sugar mill to increase capacity. As a result, this arrangement can increase the capacity of the existing sugar mill, as much as doubling it, while increasing the ethanol production over and above what would be expected with just increasing the throughput of the existing plant; producing up to three times the ethanol.

As used herein, the term “existing sugar mill” refers to a production facility that has been used as a sugar mill prior to adding a cane fractionation system or other tissue fractionation system to the facility to work in conjunction with the mill.

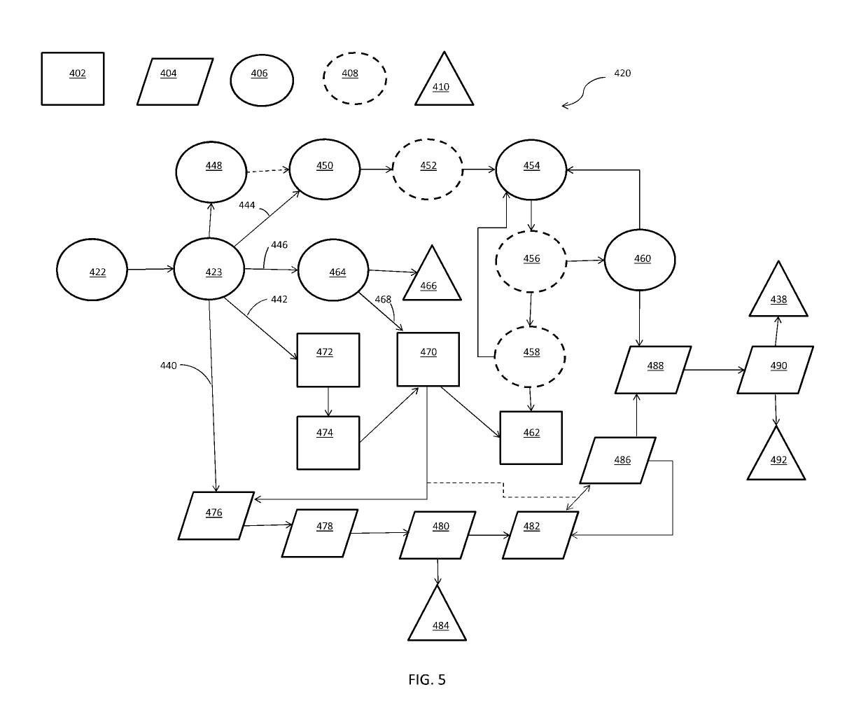

FIG. 5 is a diagram illustrating one embodiment of a tissue fractionation system installed in an existing sugar mill. In the system 420 illustrated in FIG. 5, the equipment is coded to indicate existing equipment in its original, unmodified form 402; existing equipment that has been expanded 404; new equipment 406; and possible/optional new equipment 408. Products 410 are also coded in FIG. 5 for easy identification.

In FIG. 5, lignocellulosic feedstock 422, such as billet harvested sugar cane, is fed to a cane tissue fractionation system 423. The cane tissue fractionation system 423 separates tissues of the lignocellulosic feedstock 422 into the following fractions: juice 440, rind 442, pith 444, wax cut 446, and trash 448. The trash 448, such as leaves, may either be collected and discarded, collected and fed to the boiler 462, or collected and stored for feeding to a pretreatment section 450. In the pretreatment section 450, the trash 448 can be treated either the same or similarly to the pith 444, eventually yielding sugars for fermentation into biofuel. More particularly, the pith fraction 444 and/or the trash fraction 448 may be pretreated by hydrolyzing the fraction 444, 448 with low or no acid at a temperature of at least 110° C., or at least 110° C., then further hydrolyzing the fraction 444, 448 with an enzyme cocktail, such as in a neutralization section 452 to produce sugars for fermentation in a fermentation section 454. From the fermentation section 454, the pith 444 and/or trash 448 may be passed to a fiber recovery section 456 and, as a further option, through an enzyme recovery section 458, prior to being separated between a yeast recovery section 460 and a boiler 462 within the original sugar mill.

The wax cut 446 may be sent to a wax recovery section 464 either at a third party location or in-house, to recover the wax 466. The remaining fiber, or wax rind 468, may be sent to a roll mill 470 within the original sugar mill to be burned with the bagasse.

The rind 442 may be sent to the existing cane handling equipment in the plant, namely a shredder 472, a diffuser 474, and the roll mill 470, for sugar recovery. Any remaining bagasse may be sent to the boiler 462 for power production. The sugar may be sent to the existing sucrose recovery equipment, described below, or, alternatively, the sugar may be sent directly to a fermentation section 482 since the sugar will have a higher proportion of monomeric sugars than the juice 440.

The juice 440 may be sent to the existing sucrose production train in the sugar mill, namely to a clarification section 476, followed by an evaporation section 478, and a crystallization section 480, from which sugar 484 may be recovered and/or sent to a fermentation section 482. A yeast recovery section 486 connected to the fermentation section 482 prepares the sugar for a beer well 488. The same beer well 488 may serve the fermentation section 454 and the yeast recovery section 460 used to process the pith 444 and/or trash 448. From the beer well 488, the sugar is sent to a distillation section 490 where the renewable material 438 such as ethanol and vinasse 492 may be produced.

According to some embodiments, the invention may be directed to a renewable material made according to any of the methods described herein.

According to some embodiments, the invention may be directed to a biofuel made according to any of the methods described herein. For example, a biofuel component composition may be derived from a lignocellulosic material that was separated into a plurality of fractions, including between about 30% and about 85% of a first fraction that includes primarily rind by dry weight, and between about 15% and about 70% of a second fraction that includes primarily pith by dry weight, prior to converting one or more of the fractions into a biofuel component. The biofuel component may include an alcohol, such as ethanol and/or butanol.

According to some embodiments, the invention may be directed to a biorefinery for producing biofuels. The biorefinery may include a tissue separation unit for separating tissues of a lignocellulosic feedstock, a pretreatment unit for treating at least some of the separated tissues of the lignocellulosic feedstock, and a conversion unit for producing a renewable material from the separated tissues. As explained above, the pretreatment unit may include mild conditions for breaking down or depolymerizing fractions that are easily digested, or harsh conditions for breaking down or depolymerizing hard-to-digest components. The conversion unit may include a saccharification unit and/or a fermentation unit for converting the pretreated materials into biofuels.

Alternatively or additionally, a biorefinery may include a cane tissue fractionation system added to a front end of an existing sugar mill. The capital cost of adding a cane tissue fractionation system to a sugar mill would be comparable to installing a second train in the sugar mill plus the cost of the lignocellulosic ethanol equipment. The advantages of this arrangement include: 1) processing the pith and trash only for lignocellulosic conversion may require less severe hydrolysis conditions than processing the whole lignocellulosic material; 2) the juice may be cleaner, thereby minimizing expansion of the clarification system; 3) the sugars recovered from the existing diffuser/roll mill train may be higher in monomeric sugars and that stream may be sent directly to fermentation, thereby minimizing modifications to the sucrose processing unit. Furthermore, using this type of system may increase the throughput of the existing sugar mill, thus debottlenecking the existing mill.

### Example 1

In this example, ethanol production is compared among four different methods based on the general methods illustrated in FIGS. 1-4 and described above. All four methods may be carried out using an energycane feedstock comprising leaves, rind, pith, and juice. More particularly, the results presented in this example are based on an overall composition of the energycane feedstock of 25% sucrose, 32% cellulose, 16% hemicellulose, 15% lignin, and 12% other species such as protein and ash on a dry weight basis, with the feedstock containing 70% moisture on a wet weight basis.

In the first method, based on the method shown in FIG. 1, 100 kg of the feedstock (wet weight basis) may be shredded and placed in a pretreatment reactor along with 35 g of sulphuric acid. The reactor contents may be heated to 160° C. and held at that temperature for 35 minutes. The pretreated slurry may then be cooled, transferred to another vessel, and treated with aqueous ammonia to raise the pH of the slurry to about 5. Additional treatments may be done at this stage to prepare the hydrolyzate for further processing. A cellulolytic enzyme cocktail can be added to the slurry and the temperature of the reaction maintained at 48° C. for 1 day. Next, the temperature of the mixture can be decreased to 30° C. and a yeast inoculum added to ferment the sugars into ethanol. Additional nutrients may be added at this stage. After 5 days the ethanol concentration in the beer can be measured by HPLC and the overall ethanol yield calculated. The beer may also be distilled to recover ethanol. The projected overall ethanol yield is shown in Table 1.

In the second method, 100 kg of the feedstock (wet weight basis) may be fed to an automated trash separation unit. The leaves may be recovered from the trash separation unit, while the stalks are fed to a KTC Tilby Cane Separation System (KTC Tilby Ltd. of Saanichton, British Columbia), which produces separated rind and pith (containing juice). The rind may be discarded at this stage. The pith may be combined with the leaves, and these two components can be shredded together. The shredded material may be placed in a pretreatment reactor. The reactor contents can be heated to 120° C. and held at that temperature for 35 minutes. Additional treatments may be done at this stage to prepare the material for further processing. The pretreated slurry may then be cooled, transferred to another vessel, and treated with aqueous ammonia to raise the pH of the slurry to about 5. Additional treatments may be done at this stage to prepare the hydrolyzate for further processing. A cellulolytic enzyme cocktail can be added to the slurry and the temperature of the reaction maintained at 48° C. for 1 day. Next, the temperature of the mixture may be decreased to 30° C. and a yeast inoculum can be added to ferment the sugars into ethanol. Additional nutrients may be added at this stage. After 5 days the ethanol concentration in the beer can be measured by HPLC and the overall ethanol yield calculated. The beer may also be distilled to recover ethanol. The projected overall ethanol yield is shown in Table 1.

In the third method, 100 kg of the feedstock (wet weight basis) may be fed to an automated trash separation unit. The leaves may be recovered from the trash separation unit, while the stalks are fed to a KTC Tilby Cane Separation System (KTC Tilby Ltd. of Saanichton, British Columbia), which produces separated rind and pith (containing juice). The rind may be shredded and placed in a pretreatment reactor along with 35 g of sulphuric acid. The reactor contents may be heated to 170° C. and held at that temperature for 35 minutes. The pith can be combined with the leaves, and these two components can be shredded together. The shredded material may be placed in a pretreatment reactor. The reactor contents can be heated to 120° C. and held at that temperature for 35 minutes. Additional treatments may be done at this stage to prepare the material for further processing. The pretreated slurries may then be cooled, transferred to another vessel, and treated with aqueous ammonia to raise the pH of the combined slurry to about 5. Additional treatments may be done at this stage to prepare the hydrolyzate for further processing. A cellulolytic enzyme cocktail can be added to the slurry and the temperature of the reaction maintained at 48° C. for 1 day. Next, the temperature of the mixture can be decreased to 30° C. and a yeast inoculum added to ferment the sugars into ethanol. Additional nutrients may be added at this stage. After 5 days the ethanol concentration in the beer can be measured by HPLC and the overall ethanol yield calculated. The beer may also be distilled to recover ethanol. The projected overall ethanol yield is shown in Table 1.

In the fourth method, 100 kg of the feedstock (wet weight basis) may be fed to an automated trash separation unit. The leaves may be recovered from the trash separation unit, while the stalks are fed to a KTC Tilby Cane Separation System (KTC Tilby Ltd. of Saanichton, British Columbia), which produces separated rind and pith (containing juice). The pith may be further processed by passing it through a three-roller mill to substantially extract the juice from it. Imbibition water to aid in extraction may also be added at this stage. The rind may be shredded and placed in a pretreatment reactor along with 35 g of sulphuric acid. The reactor contents can be heated to 170° C. and held at that temperature for 35 minutes. The extracted pith may be combined with the leaves, and these two components can be shredded together. The shredded material can be placed in a pretreatment reactor. The reactor contents may be heated to 120° C. and held at that temperature for 35 minutes. Additional treatments may be done at this stage to prepare the material for further processing. The pretreated slurries may then be cooled, transferred to another vessel, and treated with aqueous ammonia to raise the pH of the combined slurry to about 5. Additional treatments may be done at this stage to prepare the hydrolyzate for further processing. A cellulolytic enzyme cocktail can be added to the slurry and the temperature of the reaction maintained at 48° C. for 1 day. Next, the temperature of the mixture was decreased to 30° C., the juice was added to the mixture, and a yeast inoculum can be added to ferment the sugars into ethanol. Additional nutrients may be added at this stage. After 5 days the ethanol concentration in the beer can be measured by HPLC and the overall ethanol yield calculated. The beer may also be distilled to recover ethanol. The projected overall ethanol yield is shown in Table 1.

TABLE 1
Ethanol Production Comparison

| Figure on | Ethanol Produced from |
|---|---|
| Which Method | 100 kg Feedstock |
| is Based | (Wet Basis) |
| FIG. 1 | 5.2 kg |
| FIG. 2 | 6.7 kg |
| FIG. 3 | 9.3 kg |
| FIG. 4 | 9.7 kg |

As shown in Table 1, the three methods involving tissue separation, those shown in FIGS. 2-4, result in much greater percentages of ethanol production. Thus, the methods described herein may result in an ethanol production of at least 6.7%, or at least 9%, based on 100 kg feedstock, wet basis.

### Example 2

In this example, separation of plant tissues is examined. Whole stalks of energycane (cultivar Ho 02-113), napier grass (PI 300086), and sugarcane (cultivar CP 03-1912) were harvested from the field in the late winter. Tissues of each crop were kept separate for the ensuing steps. The stalks were stripped of leaves and leaf sheathes by hand, and the leaves and leaf sheathes were collected and weighed. The stripped stalks were cut into 1-foot segments and split in half. The pith tissue on the inside of the stalk was stripped away from the rind tissue on the outside of the stalk, and each tissue was collected separately and weighed. Each collected tissue was then milled in a Dedini cane disintegrator, available from Dedini Industrial De Base, Brazil (www.dedini.com.br). The disintegrated material was tested for moisture using a halogen moisture balance. The weights, moisture contents, and dry weights of the collected tissues are shown in Table 2.

TABLE 2
Weight and Moisture Content of Separated Plant Tissues

| | Wet | Moisture | Dry |
|---|---|---|---|
| Crop and Tissue | Weight (kg) | Content (%) | Weight (kg) |
| Ho 02-113 | | | |
| Pith | 0.784 | 73.4 | 0.209 |
| Leaves | 0.258 | 27.2 | 0.188 |
| Rind | 0.706 | 45.5 | 0.385 |
| PI 300086 | | | |
| Pith | 0.646 | 80.4 | 0.127 |
| Leaves | 0.324 | 48.8 | 0.166 |
| Rind | 1.316 | 50.0 | 0.658 |
| CP 03-1912 | | | |
| Pith | 2.80 | 71.1 | 0.809 |
| Leaves | 0.314 | 38.2 | 0.194 |
| Rind | 1.19 | 54.3 | 0.544 |

### Example 3

In this example, pretreatment of separated plant tissues is examined. Separated energycane pith and rind as prepared in Example 2 were subjected to pretreatment according to the following procedure. Wholestalk energycane prepared by milling in a Dedini cane disintegrator was also subjected to pretreatment according to the following procedure. The milled feedstock was washed with four successive volumes of deionized water at 70° C., then pressed to a consistency of approximately 30% dry solids. Next, sufficient pressed feedstock to supply 40.0 g dry weight of biomass was added to a microwave reactor vessel. Aqueous sulphuric acid (50.0 ml, 1% w/w) was added, along with enough deionized water to bring the overall dry solids loading of the pretreatment reaction mixture to 16.7%. The contents of the vessel were mixed well, and the vessel was sealed in the microwave reactor. In the microwave, the pretreatment reaction mixture was heated rapidly to 160° C. and held at that temperature for 30 minutes. The reaction mixture was then cooled to 60° C. The liquids in the reaction mixture were analyzed by HPLC, and the liquid hydrolyzate composition was reported in Table 3.

TABLE 3
Liquid Hydrolyzate Composition from
Whole and Separated Energycane

| | Cello- | Glu- | Xy- | Galac- | Arabi- | Fur- | |
|---|---|---|---|---|---|---|---|
| Biomass | biose | cose | lose | tose | nose | fural | HMF |
| source | (g/L) | (g/L) | (g/L) | (g/L) | (g/L) | (g/L) | (g/L) |
| Whole | 0.54 | 1.71 | 12.32 | 0.69 | 2.17 | 3.22 | 0.10 |
| energycane | | | | | | | |
| Energycane | 2.46 | 3.61 | 12.10 | 0.70 | 3.20 | 4.86 | 0.11 |
| pith | | | | | | | |
| Energycane | 0.49 | 3.69 | 37.81 | 1.18 | 1.83 | 2.06 | 0.31 |
| rind | | | | | | | |

### Example 4

In this example, pretreatment and enzymatic hydrolysis of separated plant tissues is examined. Separated sugarcane pith (4C, cultivar CP 03-1912), energycane rind (4B, Ho 02-113), and energycane leaves (4D, Ho 02-113) as prepared in Example 2 were subjected to pretreatment according to the following procedure. Wholestalk energycane (4A, Ho 02-113) prepared by milling in a Dedini cane disintegrator was also subjected to pretreatment according to the following procedure. The milled feedstock was washed with four successive volumes of deionized water at 70° C., then pressed to a consistency of approximately 30% dry solids. Next, sufficient pressed feedstock to supply 15.0 g dry weight of biomass was added to a microwave reactor vessel. Aqueous sulphuric acid (37.5 ml, 1% w/w) was added, along with enough deionized water to bring the overall dry solids loading of the pretreatment reaction mixture to 9.1%. The contents of the vessel were mixed well, and the vessel was sealed in the microwave reactor. In the microwave, the pretreatment reaction mixture was heated rapidly to 160° C. and held at that temperature for 30 minutes. The reaction mixture was then cooled to room temperature. The liquids in the reaction mixture were analyzed by HPLC, and the liquid hydrolyzate composition is reported in Table 4.

TABLE 4
Liquid Hydrolyzate Composition from Whole
and Separated Energycane and Sugarcane

| Biomass | Glucose | Xylose | Galactose | Arabinose | Furfural | HMF |
|---|---|---|---|---|---|---|
| source | (g/L) | (g/L) | (g/L) | (g/L) | (g/L) | (g/L) |
| Whole | 2.29 | 21.45 | 1.13 | 2.52 | 2.54 | 0.28 |
| energycane | | | | | | |
| Sugarcane | 6.28 | 18.63 | 1.28 | 2.45 | 2.56 | 1.61 |
| pith | | | | | | |
| Energycane | 1.99 | 22.03 | 0.95 | 1.96 | 2.70 | 0.34 |
| rind | | | | | | |
| Energycane | 3.22 | 20.58 | 1.21 | 3.07 | .99 | .09 |
| leaves | | | | | | |

The solids remaining in the hydrolyzed biomass were collected by filtration and washed thoroughly with water. A portion of the washed solids were enzymatically digested for 72 hours at 5% solids loading, pH 5.5, and 35° C. in a reaction mixture containing 38 μl Biocellulase W (Kerry Biosciences), β-glucosidase (1 mg/g of washed solids), 50 mM sodium citrate, 5 mM NaN₃, and 50 μg/ml kanamycin. The sugars released by enzymatic hydrolysis were measured at 24, 48, and 72 hours by HPLC. The overall sugar yields (as a percentage of the theoretical yield of sugar from the washed, pressed feedstock) from each step are reported in Table 5.

TABLE 5
Sugar Yields from Hydrolysis of Whole and Separated Energycane and Sugarcane

| | | Glucose | | | | |
|---|---|---|---|---|---|---|
| | Glucose | from | Overall | Xylose | Glucose | Xylose |
| | from acid | enzymatic | glucose | from acid | Produced | Produced |
| Biomass | hydrolysis | hydrolysis | yield | hydrolysis | (lbs/US | (lbs/US |
| source | (%) | (%) | (%) | (%) | ton) | ton) |
| Whole | 5.30 | 64.99 | 70.29 | 80.81 | 609.41 | 429.46 |
| energycane | | | | | | |
| Sugarcane | 14.64 | 64.74 | 79.37 | 68.23 | 684.76 | 374.48 |
| pith | | | | | | |
| Energycane | 4.35 | 50.33 | 54.68 | 80.74 | 504.15 | 443.30 |
| rind | | | | | | |
| Energycane | 7.89 | 67.95 | 75.84 | 83.46 | 625.60 | 415.35 |
| leaves | | | | | | |

Thus, the methods herein using pretreatment and enzymatic hydrolysis of separated plant tissues may result in a glucose yield from acid hydrolysis of at least 4%, or at least 7%, or at least 14%, and/or a glucose yield from enzymatic hydrolysis of at least 50%, or at least 64%, or at least 67%, and/or an overall glucose yield of at least 54%, or at least 75%, or at least 79%. Additionally, the methods herein using pretreatment and enzymatic hydrolysis of separated plant tissues may result in a xylose yield from acid hydrolysis of at least 68%, or at least 80%, or at least 83%.

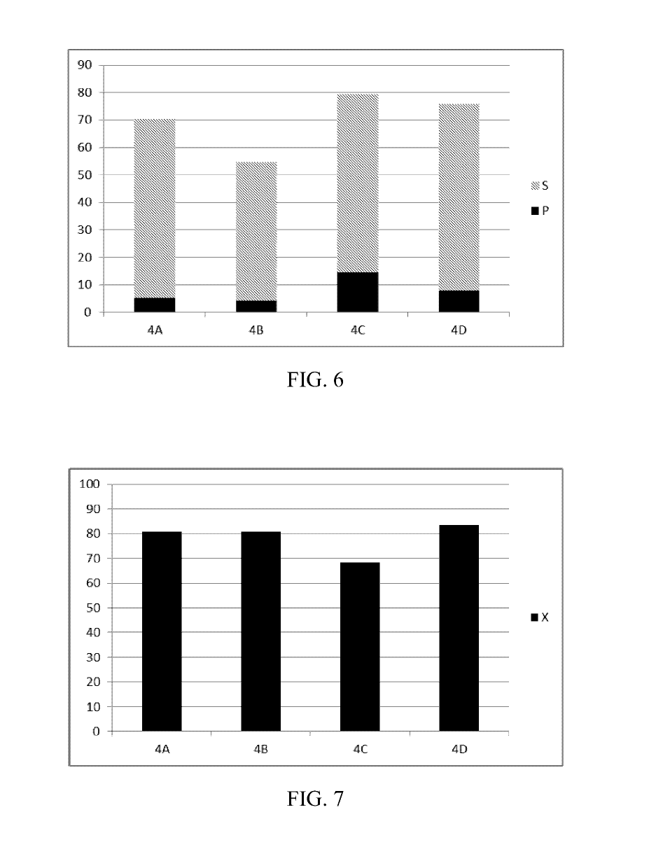

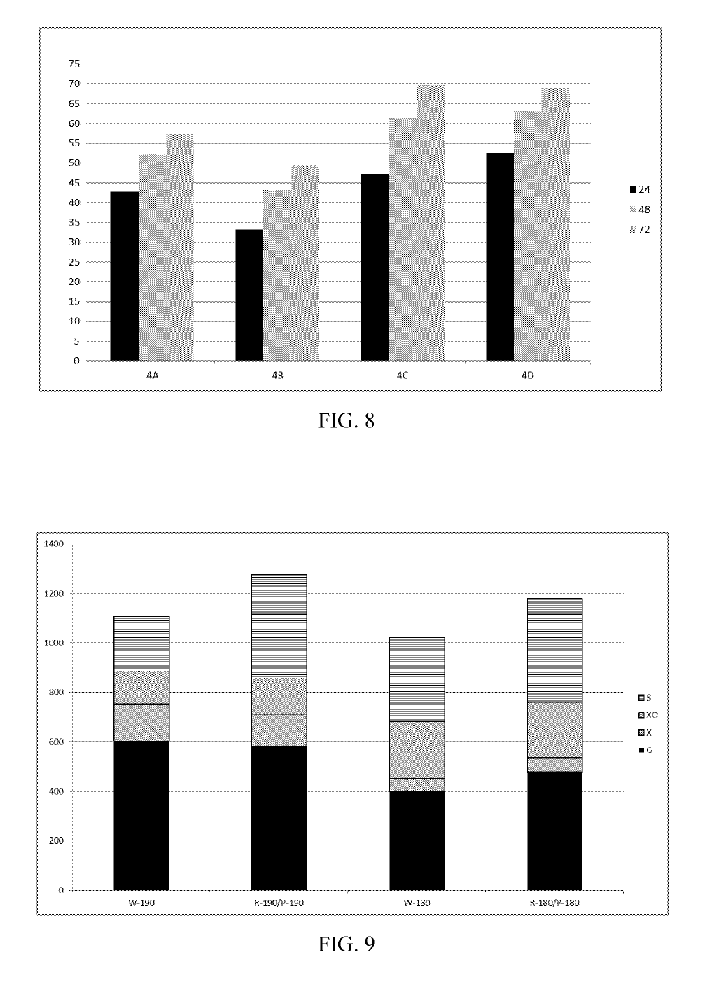

Additional results are provided in FIGS. 6-8. More particularly, FIG. 6 is a graph showing glucose molar yield from pretreatment (P) and saccharification (S) as a percentage of glucan in the washed feedstock. FIG. 7 is a graph showing xylose molar yield (X) from pretreatment as a percentage of xylan in the washed feedstock. FIG. 8 is a graph showing glucose yield at various times (24, 48, and 72 hours) in saccharification as a percentage of glucan in the pretreated solids.

### Example 5

In this example, washed and size-reduced samples of whole energycane, pith, and rind were prepared. Whole stalks of energycane (cultivar Ho02-113) were stripped of leaves (including leaf sheathes). The stalks were cut into billets 6″ to 12″ long. Some of these billets were prepared by passing them through a mechanical KTC Tilby Cane Separation System (KTC Tilby Ltd. of Saanichton, British Columbia), which produces separated rind and pith (containing juice). Each of the fractions was frozen in storage until the next steps were performed. The pith was washed with four successive volumes of deionized water at 70° C., then pressed to a consistency of approximately 30% dry solids. The rind was cut into short segments <1″ long and was size reduced to short fibers in a blender with added water. The rind fibers were washed with four successive volumes of deionized water at 70° C., then pressed to a consistency of approximately 30% dry solids. The whole energycane billets were cut into short segments <1″ long and were size reduced in a blender with added water. The resulting cane slurry was washed with four successive volumes of deionized water at 70° C., then pressed to a consistency of approximately 30% dry solids. The washed pith, rind, and whole cane samples were stored in a freezer until use.

### Example 6

In this example, pretreatment and enzymatic hydrolysis of mechanically separated plant tissues is examined. Washed pith, rind, and whole cane prepared by the methods of Example 5 were subjected to pretreatment according to the following procedure. Sufficient pressed feedstock to supply 15.0 g dry weight of biomass was added to a microwave reactor vessel. Enough deionized water was added to bring the overall dry solids loading of the pretreatment reaction mixture to 9.1%. The contents of the vessel were mixed well, and the vessel was sealed in the microwave reactor. In the microwave, the pretreatment reaction mixture was heated rapidly to 180° C. or 190° C. depending on the entry and held at that temperature for 30 minutes. The reaction mixture was then cooled to room temperature. The liquids in the reaction mixture were analyzed by HPLC, and the liquid hydrolyzate composition are reported in Table 6.

TABLE 6
Liquid Hydrolyzate Composition from Whole
and Separated Energycane and Sugarcane

| | | Glucose & | Xylose & | | | | |
|---|---|---|---|---|---|---|---|
| | Pretreatment | Gluco- | Xylo- | | | | |
| Biomass | Temperature | oligomers | oligomers | Galactose | Arabinose | Furfural | HMF |
| source | (° C.) | (g/L) | (g/L) | (g/L) | (g/L) | (g/L) | (g/L) |
| Whole | 180 | 1.22 | 19.69 | 0.08 | 1.02 | 0.02 | 0.49 |
| energycane | | | | | | | |
| Energycane | 180 | 0.39 | 21.35 | 0.10 | 0.97 | 0.01 | 0.49 |
| rind | | | | | | | |
| Energycane | 180 | 2.45 | 18.72 | 0.28 | 1.18 | 0.02 | 0.68 |
| pith | | | | | | | |
| Whole | 190 | 1.25 | 20.17 | 0.30 | 1.46 | 0.06 | 1.87 |
| energycane | | | | | | | |
| Energycane | 190 | 0.49 | 19.40 | 0.13 | 1.38 | 0.04 | 1.86 |
| rind | | | | | | | |

The solids remaining in the hydrolyzed biomass were collected by filtration and washed thoroughly with water. A portion of the washed solids were enzymatically digested for 72 hours at 5% solids loading, pH 5.0, and 50° C. in a reaction mixture containing Biocellulase TR1 (76 μl, Kerry Biosciences), β-glucosidase (2 mg/g of washed solids), 50 mM sodium acetate, 5 mM NaN₃, and 50 μg/ml kanamycin. The sugars released by enzymatic hydrolysis were measured at 24, 48, and 72 hours by HPLC. The overall sugar yields (as a percentage of the theoretical yield of sugar from the washed, pressed feedstock) from each step are reported in Table 7.

TABLE 7
Sugar Yields from Hydrolysis of Whole and Separated Energycane and Sugarcane

| | | | | | | Xylose & | | |
|---|---|---|---|---|---|---|---|---|
| | | | Glucose | | | xylo- | | Xylose & |
| | | Glucose | from | Overall | Xylose | oligomers | Glucose | Oligomers |
| | | from pre- | enzymatic | glucose | from pre- | from pre- | Produced | Produced |
| Biomass | Temp | treatment | hydrolysis | yield | treatment | treatment | (lbs/US | (lbs/US |
| source | (° C.) | (%) | (%) | (%) | (%) | (%) | ton) | ton) |
| Whole | 180 | 2.58 | 50.36 | 52.93 | 11.64 | 64.56 | 526 | 372 |
| energycane | | | | | | | | |
| Energycane | 180 | 1.20 | 48.91 | 50.11 | 12.94 | 63.88 | 520 | 375 |
| rind | | | | | | | | |
| Energycane | 180 | 1.52 | 71.09 | 72.61 | 13.31 | 64.14 | 804 | 371 |
| pith | | | | | | | | |
| Whole | 190 | 2.44 | 77.01 | 79.45 | 34.09 | 65.06 | 792 | 375 |
| energycane | | | | | | | | |
| Energycane | 190 | 2.01 | 69.57 | 71.58 | 38.55 | 62.10 | 738 | 365 |
| rind | | | | | | | | |

Thus, the methods herein using pretreatment and enzymatic hydrolysis of mechanically separated plant tissues may result in a glucose yield from pretreatment of at least 1.2%, or at least 1.5%, or at least 2%, and/or a glucose yield from enzymatic hydrolysis of at least 48%, or at least 69%, or at least 71%, and/or an overall glucose yield of at least 50%, or at least 71%, or at least 72%. Additionally, the methods herein using pretreatment and enzymatic hydrolysis of mechanically separated plant tissues may result in a xylose yield from pretreatment of at least 12%, or at least 13%, or at least 38%, and/or a xylose and xylooligomers yield from pretreatment of at least 62%, or at least 63%, or at least 64%.

Additional results are provided in FIG. 9. More particularly, FIG. 9 shows Soluble sugars (S), Glucose (G), xylose (X), and xylo-oligomers (XO) estimated to be produced by extraction, pretreatment, and saccharification in lbs per US dry ton feedstock separated and pretreated by different methods. W-190=Whole energycane pretreated at 190° C.; R-190/P-180=Energycane separated into rind (190° C. pretreatment) and pith (180° C. pretreatment); W-180=Whole energycane pretreated at 180° C.; R-190/P-180=Energycane separated into rind (180° C. pretreatment) and pith (180° C. pretreatment).

### Example 7

In this example, enzymatic hydrolysis of mechanically separated plant tissues is examined. Washed pith (7C), rind (7B), and whole cane (7A) prepared by the methods of Example 5 were subjected to enzymatic hydrolysis for 72 hours at 5% solids loading, pH 5.5, and 35° C. in a reaction mixture containing Biocellulase TR1 (38 μl, Kerry Biosciences), β-glucosidase (1 mg/g of washed solids), 50 mM sodium citrate, 5 mM NaN₃, and 50 μg/ml kanamycin. The sugars released by enzymatic hydrolysis were measured at 24, 48, and 72 hours by HPLC. The overall sugar yields at 72 hours (as a percentage of the theoretical yield of sugar from the washed, pressed feedstock) are reported in Table 8.

TABLE 8
Sugar Yields from Hydrolysis of Whole
and Separated Energycane and Sugarcane

| | | | Glucose | Xylose |
|---|---|---|---|---|
| Biomass | Glucose | Xylose | Produced | Produced |
| source | (%) | (%) | (lbs/US ton) | (lbs/US ton) |
| Whole | 13.1 | 2.7 | 128 | 16 |
| energycane | | | | |
| Energycane | 4.7 | 1.5 | 48 | 8 |
| rind | | | | |
| Energycane | 29.9 | 3.9 | 317 | 22 |
| pith | | | | |

Thus, the methods herein using enzymatic hydrolysis of mechanically separated plant tissues may result in a glucose yield of at least 4%, or at least 29% and/or a xylose yield of at least 1.5%, or at least 3.9%.

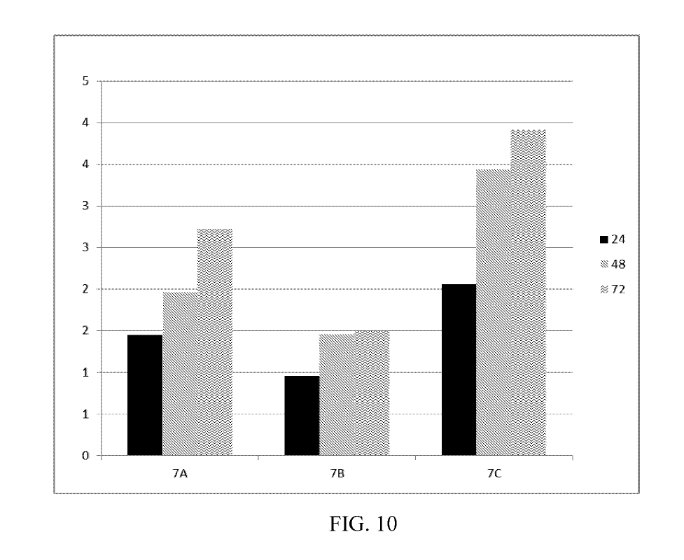

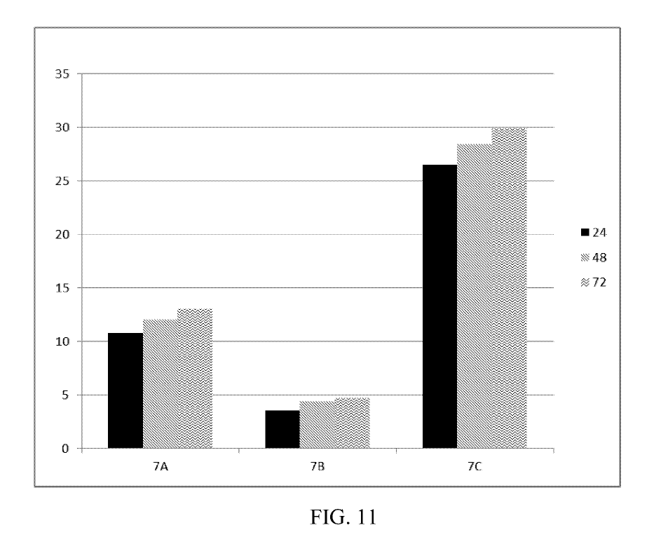

Additional results are provided in FIGS. 10 and 11. More particularly, FIG. 10 shows xylose yield at various times (24, 48, and 72 hours) in saccharification as a percentage of xylan in the washed feedstocks. FIG. 11 shows glucose yield at various times (24, 48, and 72 hours) in saccharification as a percentage of glucan in the washed feedstocks.

It will be apparent to those skilled in the art that various modifications and variations can be made in the disclosed structures and methods without departing from the scope or spirit of the invention. Particularly, descriptions of any one embodiment can be freely combined with descriptions or other embodiments to result in combinations and/or variations of two or more elements or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

1-103. (canceled)

104. A method of producing a renewable material, the method comprising:
mechanically separating lignocellulosic feedstock into at least a first fraction comprising rind and a second fraction comprising pith;
pretreating at least one of the fractions; and
further treating at least one pretreated fraction to produce a renewable material.

105. The method of claim 104, comprising separating the lignocellulosic feedstock into at least a first fraction, a second fraction, and a third fraction.

106. The method of claim 104, comprising separating the lignocellulosic feedstock and subsequently extracting juice from the lignocellulosic feedstock.

107. The method of claim 104, further comprising:
generating a first fraction enriched in water and a second fraction depleted in water; and
combusting the fraction depleted in water.

108. The method of claim 104, comprising:
pretreating a fraction comprising rind using a harsh pretreatment process; and
pretreating a fraction comprising pith and/or leaves using a mild pretreatment process.

109. The method of claim 104, wherein the pretreating step comprises using an acid selected from the group consisting of sulfuric acid, hydrochloric acid, sulfonic acid, phosphoric acid, nitric acid, acetic acid, lactic acid, formic acid, oxalic acid, and combinations thereof.

110. The method of claim 104, wherein the pretreatment is carried out without heating the at least one fraction.

111. The method of claim 104, further comprising:
converting at least one of the fractions to one or more sugars; and
converting the sugars into a renewable material.

112. The method of claim 104, wherein the renewable material comprises ethanol.

113. The method of claim 104, wherein the renewable material comprises material suitable for use as biofuels, blendstocks, Chemicals, intermediates, solvents, adhesives, polymers, and/or lubricants.

114. A method of producing a renewable material, the method comprising:
separating lignocellulosic feedstock into the following fractions: juice, rind, pith, wax cut, and trash;
pretreating at least one of the fractions; and
further treating the at least one pretreated fraction to produce a renewable material.

115. The method of claim 114, comprising:
using a cane tissue fractionation system at a front end of an existing sugar mill to separate the lignocellulosic feedstock into the fractions;
sending the juice fraction to a sucrose production train as part of the existing sugar mill;
sending the rind fraction to cane handling equipment for sugar recovery as part of the existing sugar mill;
sending a bagasse remainder to a boiler for power production; and
sending the sugar either to a sucrose recovery unit or to a fermentation unit.

116. The method of claim 114, further comprising:
pretreating the pith fraction by hydrolyzing the pith fraction with low or no acid at a temperature of at least 110° C., then
further hydrolyzing the pith fraction with an enzyme cocktail to produce sugars for fermentation; and
sending wax from the wax cut fraction to a wax recovery unit.

117. A renewable material made according to claim 104.

118. A biorefinery for producing biofuels, comprising:
a cane tissue fractionation system at a front end of the biorefinery;
a sugar mill behind the cane tissue fractionation system; and
a conversion unit for producing a renewable material;
wherein the cane tissue fractionation system separates tissues of the lignocellulosic feedstock into the following fractions: juice, rind, pith, wax cut, and trash.
